(19) United States
(12) Patent Application Publication
(10) Pub. No.: US 2010/0049098 A1
(43) Pub. Date: Feb. 25, 2010

(54) Automatic acoustic treatment device

(76) Inventors: Avi Shalgi, Tel Aviv (IL); Igor Nudelman, Herzliya (IL); Avner Yanai, Highland Park, IL (US); Leonid Kushculey, Rehovot (IL)

Correspondence Address: FENNEMORE CRAIG, 3003 NORTH CENTRAL AVENUE, SUITE 2600, PHOENIX, AZ, 85012, US

(21) Appl. No.: 12/222,960

(22) Filed: Aug. 20, 2008

(51) Int. Cl. A61N 7/00

(52) U.S. Cl. 601/2

(57) ABSTRACT

There is provided herein an apparatus for lysing of adipose tissue, comprising a transducer adapted to transmit ultrasound acoustic waves to a target area tissue of a subject body and a controller adapted to automatically trigger the transducer to transmit ultrasound acoustic waves upon receiving indication of said transducer being positioned at a predetermined position (node). There is further provided herein an apparatus for lysing of adipose tissue, comprising a transducer adapted to transmit ultrasound acoustic waves to a target area tissue of a subject body and a positioning element adapted to automatically position the transducer at a predetermined position (node).

### FIELD

The invention relates to devices for performing acoustic treatments on tissue.

### BACKGROUND

Aesthetic medicine, a relatively new and rapidly growing field in medicine, is a form of medical practice devoted to promoting aesthetic traits in people. Among many areas covered by aesthetic medicine, “body contouring” is frequently regarded as one of the more popular. Body contouring comprises reshaping parts of a body by removing and/or reducing subcutaneous fat cells and adipose tissue in different parts of the body, using medical procedures and/or devices which may be invasive or non-invasive.

A form of non-invasive destruction of adipose (fat) tissue which may be used in body contouring includes delivering focused energy, for example, ultrasound, to a region of tissue, to perform therapeutic and/or cosmetic procedures on a patient's tissue. Generally, tissue destruction may be performed using focused energy, such as, for example, focused ultrasound (FU) energy, which can cause tissue damage by two main mechanisms, namely, thermal and mechanical mechanisms.

As referred to herein, the term Focused Ultrasound (FU) is related to ultrasonic energy that may be externally and non-invasively applied to a surface in a focused manner such that the energy is focused to a specified internal target area. The ultrasonic (acoustic) energy applied may be, for example, in the form of waves. Applying and focusing the ultrasonic energy may be performed by various ways, such as, for example, by an ultrasonic transducer that may be adapted to focus the ultrasonic energy. The focused ultrasonic energy may include various intensity levels. For example, FU may include High Intensity Focused Ultrasound (known in the art as HIFU). For example, the FU may be applied to/on a subject skin and focused on a subcutaneous target area/volume, such as fat cells and adipose tissue.

As referred to herein, the term “HIFU” relates to High Intensity Focused Ultrasound—the use of high intensity focused ultrasound energy in ultrasound treatment (therapy). According to some embodiments, the term HIFU may further encompass MIFU and/or LIFU.

As referred to herein, the term “MIFU” relates to Mid Intensity Focused Ultrasound—the use of medium intensity focused ultrasound energy in ultrasound treatment.

As referred to herein, the term “LIFU” relates to Low Intensity Focused Ultrasound—the use of low intensity focused ultrasound energy in ultrasound treatment.

The thermal mechanism includes an increase of temperature (heating) within the treated area, obtained by a direct absorption of ultrasonic energy by the treated tissue. The increased temperature causes damaging processes, such as coagulation, within the tissue. The mechanical mechanism mainly includes streaming, shear forces, tension and cavitation, which is the formation of small bubbles within the tissue. These processes cause fractionation, rapture and/or liquefaction of cells, which, in turn, results in tissue destruction. Cavitation is a physical phenomenon in which low-pressure bubbles are formed and then tend to collapse in a liquid. Cavitation near cells will damage or destroy many of the cells. The cavitation phenomenon depends on specific tissue characteristics when employed in a biological environment. This enables tissue differentiation for damage or destruction, which means that fat cells can be destroyed (or damaged sufficiently to die soon after), while blood vessels, peripheral nerves, skin, muscle and connective tissue within the ultrasonic focus, as well as neighboring tissues such as listed above outside the focus, will remain intact.

Other destructive mechanisms, such as cell apoptosis, may also directly or indirectly be involved in the non-invasive ultrasonic treatment.

Body contouring techniques comprising the use of focused ultrasound (FU) generally require relatively long treatment times. During this time, the patient is usually lying down while a treatment provider moves a device, comprising an acoustic transducer, from one position (node) to another within a treatment area on the patient's body. The provider must stop the device at every node, which may be positioned a few millimeters, and sometimes centimeters, from one another, and trigger the transducer to emit one or more acoustic pulses substantially in a right place.

Some focused ultrasound body contouring treatments require the provider to cover a relatively large number of nodes, for example 500-1500 nodes, as may be in some cases when the treatment area includes the abdominal area. The treatment, depending on the technique used, may be rather lengthy, requiring, in some cases, 90 minutes and sometimes even more, which may be uncomfortable for the patient and inefficient for the doctor. Additionally, the lengthy treatment time may result in a decreased device utilization rate (number of treatments performed with the device per period of time) resulting in a relatively high treatment cost to the patient.

A possible solution is to increase the distance between the nodes, but this may also reduce treatment efficacy.

### SUMMARY

An aspect of some embodiments of the invention relates to providing an apparatus for lysing of adipose tissue adapted to provide relatively short treatment time. Shortened treatment time may result from a number of aspects. Among these aspects are, for example, shorter node time and shorter time between nodes (Thn).

Energy at each node may be radiated from the apparatus in a continuous wave (CW) mode, or in a pulsed mode comprising tone bursts. The energy radiated at the node in a pulsed mode generally comprises bursts, which may be characterized by a tonal frequency, f; a period T=1/f; a burst length, Ton; a burst repetition period, Tbrp; a burst repetition frequency BRF=1/Tbrp; a duty cycle, DC=Ton/Tbrp; and a pulse duration or treatment time per node, Tn. Shortening node time (Tn), while essentially maintaining the applied energy, may be accomplished, for example, by increasing ultrasound burst repetition frequency (BRF) (such as, but not limited to, by a factor of three, to 75 Hz instead of 25 Hz). Shortening node time (Tn), while essentially maintaining the applied energy, may also be accomplished, for example, by increasing duty cycle, DC, (such as, but not limited to, 1:7 instead of 1:21). Shortening node time (Tn), while essentially maintaining the applied energy, may also be accomplished, for example, by increasing the applied acoustic power (such as, but not limited to 300 W instead of 100 W). Of course, any combination of the above-mentioned parameters may be applied to shortening node time while essentially maintaining the applied energy. Depending on the selected parameters, thermal and/or cavitational effects may be increased.

An aspect of some embodiments of the invention relates to providing an apparatus for lysing bf adipose tissue; the apparatus includes a transducer adapted to transmit ultrasound to a target area tissue of a subject body and a controller adapted to automatically trigger the transducer to transmit ultrasound upon receiving indication of said transducer being positioned at a predetermined position (such as a node). According to an aspect of some embodiments of the invention, the apparatus is adapted to automatically trigger transducer pulses when the transducer is correctly positioned above a node. This may allow saving the time that passes between receiving (by doctor or any other personnel) an indication that the transducer is correctly positioned and manually activating the transducer to transmit ultrasound. Accordingly, the duration of the treatment may be shortened while still maintaining the desired efficacy.

Furthermore, according to an aspect of some embodiments of the invention, the transducer may be adapted to transmit pulses having an increased duty cycle, for example, 1:7 instead of 1:20, allowing for a same amount of acoustic energy to be delivered over a shorter period of time.

Another aspect of some embodiments of the invention relates to providing an apparatus for lysing of adipose tissue; the apparatus includes a transducer adapted to transmit ultrasound to a target area tissue of a subject body and positioning element (such as a “robot” arm) adapted to automatically position the transducer according to data obtained by a tracking system. In this automatic mode of operation, the transducer automatically moves from node to node, generally following a predetermined path within a node map.

Another aspect of some embodiments of the invention relates to providing an apparatus for lysing of adipose tissue; the apparatus includes a transducer adapted to transmit ultrasound to a target area tissue of a subject body and a tracking system.

The tracking system is adapted to identify the location of the transducer or a certain spot on the transducer. The tracking system may also be adapted to iteratively guide the transducer through a node map one or more times until an indication is obtained that a certain node has received a predetermined amount of acoustic energy. Nodes which have received the predetermined amounts of acoustic energy are bypassed by the transducer during subsequent iterations.

The node map may be prepared, for example, by marking (visibly or non-visibly) on a subject's body, in the treatment area, a contour map, specific points or any other marking. The node map may also be prepared by optically marking the treatment area using an optical reader such as an “optical pen”, a laser scanner, a CCD reader, a camera, or any other device adapted to optically record the treatment area.

The tracking system may include any tracking and/or guidance system such as, but not limited to, an electromagnetic tracking system, acoustic tracking system, optical tracking system and/or an imaging system, which may include, for example, ultrasound imaging, video imaging, or other forms of imaging. In an embodiment of the invention, the tracking system may be a part of the transducer (integral part or an affixable part). In an embodiment of the invention, the tracking system may be physically distant from the transducer and have the means to track the position of the transducer. Optional tracking systems may include, CAT (computer-aided tomography), MRI (magnetic resonance imaging), and PET (positron emission tomography) or any other appropriate tracking means.

Digital processing of a marked treatment area may facilitate determination of a relatively exact position for each node in the node map. In an embodiment of the invention, the node map and/or the nodes may be displayed in a display comprised in the treatment device adapted to show the position of each node within the treatment area. The display may be further adapted to show a status of applied acoustic energy to the node (whether acoustic energy has been applied to the node or not, optionally partly or wholly).

In another embodiment of the invention, positioning of the transducer is performed by a robot arm guided by a user (such as a physician or a user). In this “semi” automatic apparatus, the user guides the robot arm, and thereby the transducer, from node to node following the node map. Optionally, positioning of the transducer is “manually” performed by the user, who moves the transducer from node to node, following the node map.

According to some embodiments of the invention, at least two modes of operation may be applied:

1. A “continuous moving of the transducer” mode of operation, which includes continuously moving the transducer from node to node, for example, but not limited to, at a rate of 1-20 mm per second. This can be done by a user or automatically by a support arm, such as a robot arm. In this mode of operation, the transducer is adapted to transmit ultrasonic energy and to continuously move along a predetermined path while transmitting the ultrasonic energy. In one embodiment, the speed (S) of moving the transducer may be calculated according to formula (1):

S=x/t (1)

wherein x represents a characteristic dimension of a node(s) (such as but not limited to, a distance between two nodes) and t represents a required node time.

2. A “discrete node” mode of operation, which includes moving the transducer from node to node and transmitting ultrasonic acoustic energy only upon reaching a certain node. When the transducer is moving from one node to another, no transmission takes place. This mode of operation can be performed by a user or automatically by a support arm, such as a robot arm.

According to some embodiments of the invention, the two modes of operation disclosed above, or any other mode of operation, may be combined. For example a treatment may start by a “continuous moving of the transducer” mode of operation and shift at a certain point to a “discrete node” mode of operation, or vice versa. This can be applied multiple times during one treatment.

There is provided, in accordance with an embodiment of the invention, an apparatus for lysing of adipose tissue comprising a transducer adapted to transmit ultrasound to a target area tissue of a subject body; and a controller adapted to automatically trigger the transducer to transmit ultrasound upon receiving indication of the transducer being positioned at a predetermined position (node). Optionally, indication of the transducer being positioned at a predetermined position is provided by a tracking system. Additionally or alternatively, the tracking system comprises a node map.

In some embodiments of the invention, the apparatus further comprises a positioning element adapted to automatically position the transducer at the predetermined position. Optionally, the positioning element comprises a robot arm. Optionally, the positioning element provides at least two degrees of freedom of movement.

In accordance with some embodiments of the invention, the transducer is adapted to transmit ultrasound with a duty cycle in the range of 1:3-1:400. Optionally, the transducer is adapted to transmit ultrasound at a pulse repetition frequency (PRF) range of 20-1100 Hz.

There is provided, in accordance with an embodiment of the invention, an apparatus for lysing of adipose tissue comprising a transducer adapted to transmit ultrasound to a target area tissue of a subject body; and a positioning element adapted to automatically position the transducer at a predetermined position (node). Optionally, the apparatus further comprises a controller adapted to notify a treatment provider upon receiving indication of the transducer being positioned at a predetermined position (node). Optionally, the apparatus further comprises a controller adapted to automatically trigger the transducer to transmit ultrasound upon receiving indication of the transducer being positioned at a predetermined position.

In some embodiments of the invention, the positioning element comprises a robot arm. Optionally, the positioning element provides at least two degrees of freedom of movement. Optionally, the positioning element is adapted to continuously move the transducer at a predefined speed. Optionally, the rate of movement is adapted to vary during a treatment. Additionally or alternatively, the positioning element is adapted to continuously move the transducer in accordance with a predetermined path.

In some embodiments of the invention, the apparatus further comprises a processor adapted to compute the amount of ultrasonic energy transmitted to a node of interest. Optionally, the apparatus further comprises a controller adapted to initiate the positioning element to return to a specific node upon receiving an indication that the amount of ultrasonic energy transmitted to the specific node is below a predetermined threshold. Optionally, the positioning element is adapted to automatically position the transducer at a predetermined position based on data obtained by a tracking system. Additionally or alternatively, the tracking system comprises a node map.

There is provided, in accordance with some embodiments of the invention, a system for lysing of adipose tissue, comprising a transducer adapted to transmit ultrasound to a target area tissue of a subject body; a positioning element adapted to automatically position the transducer at a predetermined position; and a controller adapted to automatically trigger the transducer to transmit ultrasound upon receiving indication of the transducer being positioned at the predetermined position.

There is provided, in accordance with an embodiment of the invention, a method for lysing of adipose tissue comprising positioning an ultrasonic transducer at a predetermined position on or in proximity to a treatment area of a subject body; and automatically triggering the transducer to transmit ultrasound upon receiving indication of the transducer being at the predetermined position (node). Optionally, positioning the ultrasonic transducer comprises manually positioning. Additionally or alternatively, positioning the ultrasonic transducer comprises automatically positioning.

In some embodiments of the invention, the method further comprises continuously moving the transducer at a predefined rate. Optionally, the rate of movement is adapted to vary during a treatment. Optionally, the method further comprises continuously moving the transducer in accordance with a predetermined path.

In accordance with some embodiments of the invention, the method further comprises computing the amount of ultrasonic energy transmitted to a node of interest. Optionally, the method further comprises returning to a specific node upon receiving indication that the amount of ultrasonic energy transmitted to the specific node is at or below a predetermined threshold and automatically triggering the transducer to transmit ultrasound.

There is provided, in accordance with an embodiment of the invention, a method for lysing of adipose tissue comprising positioning an ultrasonic transducer at a predetermined position (node) on or in proximity to a treatment area of a subject body; triggering the transducer to transmit ultrasound upon receiving indication of the transducer being at the predetermined position (node); computing the amount of ultrasonic energy transmitted to a node of interest; and returning to a specific node upon receiving indication that the amount of ultrasonic energy transmitted to the specific node is at or below a predetermined threshold and triggering the transducer to transmit ultrasound. Optionally, positioning the ultrasonic transducer comprises manually positioning. Additionally or alternatively, positioning the ultrasonic transducer comprises automatically positioning. Optionally, triggering comprises automatic triggering.

### BRIEF DESCRIPTION OF FIGURES

Examples illustrative of embodiments of the invention are described below with reference to figures attached hereto. In the figures, identical structures, elements or parts that appear in more than one figure are generally labeled with a same numeral in all the figures in which they appear. Dimensions of components and features shown in the figures are generally chosen for convenience and clarity of presentation and are not necessarily shown to scale. The figures are listed below.

FIG. 1A schematically shows an exemplary automatic acoustic treatment device for tissues (apparatus), in accordance with an embodiment of the invention;

FIG. 1B schematically shows an exemplary robot arm, in accordance with an embodiment of the invention;

FIG. 2 schematically shows an exemplary automatic apparatus, in accordance with another embodiment of the invention;

FIG. 3 schematically shows an exemplary manual apparatus, in accordance with another embodiment of the invention;

FIG. 4 schematically shows an exemplary manual apparatus, in accordance with another embodiment of the invention;

FIG. 5 shows a flowchart of a mode of operation of the apparatus shown in FIG. 1 or FIG. 2, in accordance with an embodiment of the invention;

FIG. 6 shows a flowchart of a mode of operation of the apparatus shown in FIG. 1 or FIG. 2, or optionally the apparatus shown in FIG. 3 or FIG. 4, in accordance with an embodiment of the invention;

FIGS. 7A-7I schematically show exemplary raster and non-raster patterns which may be followed by the transducer shown in FIG. 1, and optionally the transducers of FIGS. 2, 3 and/or 4, when moving throughout the treatment area, in accordance with some embodiments of the invention; and

FIG. 8 schematically shows an exemplary ultrasonic acoustic energy waveform transmitted by the transducer shown in FIG. 1, and optionally the transducers of FIGS. 2, 3 and/or 4, in accordance with some embodiments of the invention.

### DETAILED DESCRIPTION

### Glossary

The term “node” may include, according to some embodiments, a position of a transducer. The position of a transducer may generally relate to a location in a treatment area that may receive acoustic energy.

The term “node map” may include, according to some embodiments, a marking that lays out the nodes, generally the nodes that are designated to be treated. The node map may be visible or virtual. The node map may be on or in proximity to a treatment area of a subject body, and/or optionally on a display.

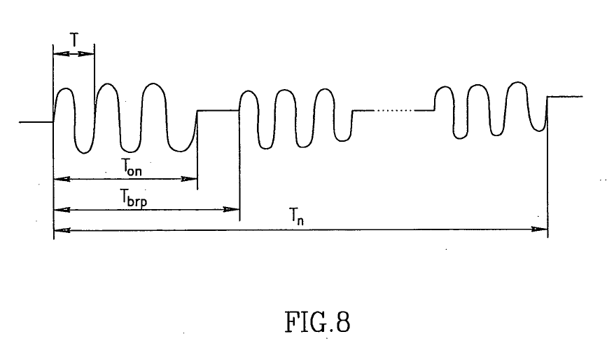

The term “node time” (Tn) may include, according to some embodiments, the length of time elapsed from the moment a transducer starts to transmit acoustic energy to a node until the transducer ceases to transmit acoustic energy to the node; that is, the treatment time per node (or transmission time, or exposure time) (also see FIG. 8). According to some embodiments of the invention, node time may be synonymous with “pulse duration”.

The term “time between nodes” (Thn) may include, according to some embodiments, the length of time elapsed from the moment transmission of acoustic energy to a first node is ceased and the transducer is ready to be moved to a next node, until the transducer is positioned to transmit acoustic energy to the next node.

The term “duty cycle” (DC) may include, according to some embodiments, the ratio of burst duration (burst length) to the time between two successive bursts (also see FIG. 8).

The term “burst repetition period” (Tbrp) may include, according to some embodiments, a time between two successive bursts (also see FIG. 8).

The term “burst repetition frequency” (BRF) may include, according to some embodiments, an inverse (reciprocal) of the time between two successive bursts (also see FIG. 8).

The term “applied power” may include, according to some embodiments, the input electric power applied to a transducer.

The term “continuously move the transducer” may include, according to some embodiments, constantly, intermittently, at a constant rate and/or at a varying rate.

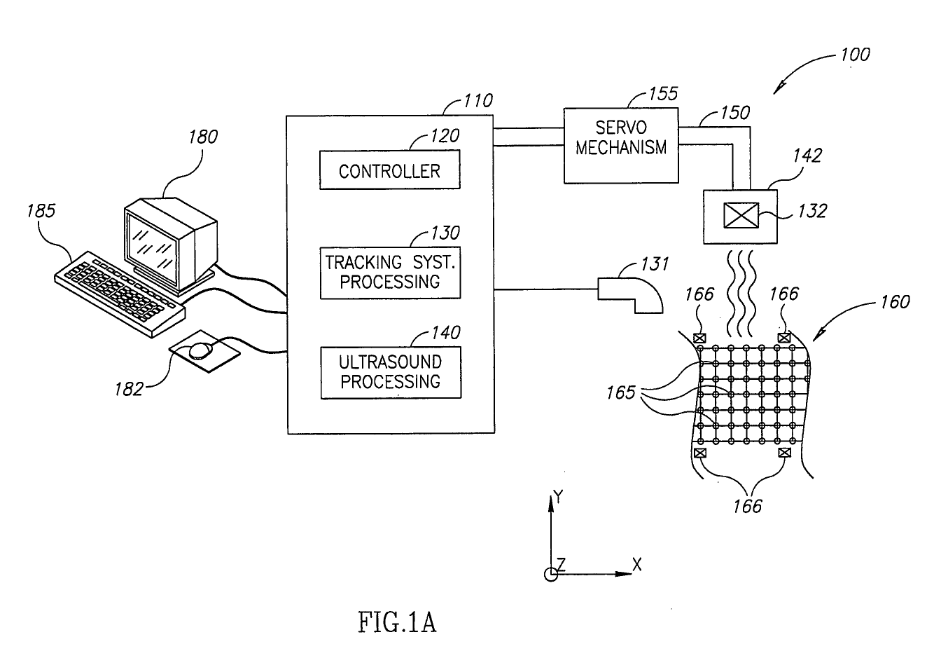

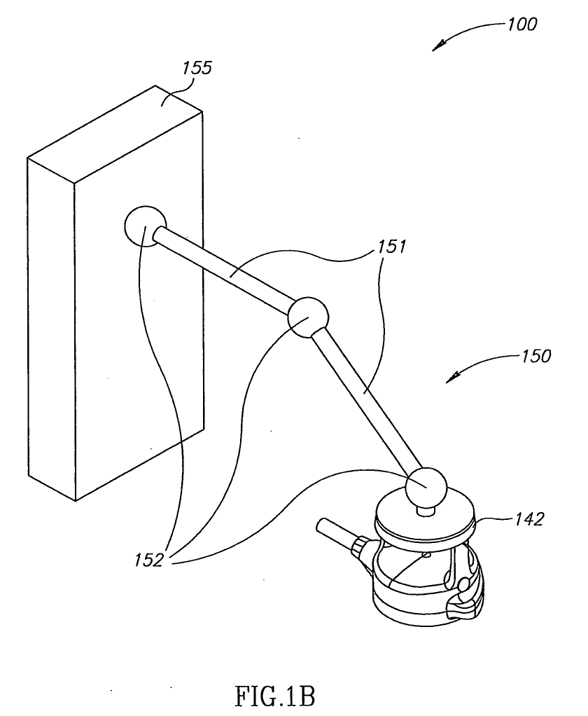

Reference is made to FIG. 1A, which schematically shows an exemplary automatic apparatus 100, in accordance with an embodiment of the invention. Apparatus 100 comprises a base unit 110 which includes a controller 120, a tracking system processor 130 and a focused ultrasound processing module 140; a transducer 142 which connects to base unit 110 through a robot arm 150; a servomechanism 155; an optional display 180; optional data input means such as a keyboard 185 and a mouse 182; a tracking system 132 comprised in, or in close proximity to, transducer 142; and an optional optical reader 131. In some embodiments of the invention, tracking system processor 130 may be comprised in controller 120.

Transducer 142 may comprise one or more transducer elements adapted to transmit focused energy, for example focused ultrasound (FU), to nodes 165 in a treatment area 160 on a patient's body.

FU processing module 140, responsive to a trigger signal from controller 120, is adapted to transmit a signal to transducer 142 to transmit focused energy. FU processing module 140 may comprise a high power pulse generator (not shown) adapted to send an electric power signal to transducer 142, which is converted by transducer 142 into acoustic energy. FU processing module 140 may also include means to cool transducer 142 and additional components as may be required to ensure effective FU transmission to nodes 165 in treatment area 160.

In accordance with an embodiment of the invention, tracking system 132 is adapted to acquire data indicative of treatment area 160 and to transmit relating data to tracking system processor 130 for processing and for building a node map of the treatment area. The node map comprises a general layout of the position of each node 165 in the treatment area 160, allowing the nodes to be referenced by their location within the node map. Tracking system 132 is further adapted to provide real-time indication of nodes 165 which, when processed by tracking system processor 130, allow for real-time tracking of the position of transducer 142 within the node map. The processed real-time information further allows for correct positioning of the transducer over the nodes, prior to automatic triggering of one or more pulses of acoustic energy. Tracking system 132 may comprise an optical tracking and guidance system, an electromagnetic (EM) guidance and tracking system, an acoustic tracking and guidance system, an IR guidance and tracking system, a laser guidance and tracking system, or any other system adapted to track and guide transducer 142 over treatment area 160, or any combination thereof. For example, tracking system 132 may comprise an optical imaging system adapted to perform three dimensional (3D) imaging of treatment area 160, or, optionally, two dimensional imaging (2D), using techniques known in the art for laser imaging, ultrasound imaging, video imaging, and/or other forms of optical imaging.

In some embodiments of the invention, treatment area 160 may be defined by reference markers 166 positioned intermittently, or, optionally continuously, along a border of the treatment area. Reference markers 166 may be the same or different from each other. In case reference markers 166 are the same, the system (for example tracking system 132) may identify them and assign them different specifications. Additionally or alternatively, markers 166 may be placed at corners of treatment area 160, or optionally, inside the treatment area, or any combination thereof. Markers 166 may be of a passive type. Optionally, markers 166 may be active and may comprise sensors adapted to interact with tracking system 132, such as, for example, light emitting sensors, EM sensors, acoustic sensors, or other types of sensors adapted to serve as reference positions for tracking system 132, or any combination thereof. According to some embodiments, treatment area 160 may include a physical node map (not shown) drawn or attached to a treated subject's body. Such physical node map may be used in combination with a tracking system 132 (which may be, for example, a camera) to provide reference of the position of transducer 142 relative to the treatment area 160. Optionally, in some embodiments of the invention, the node map may be built using manual optical reader 131. Prior to start of treatment, optical reader 131 may be adapted to acquire images of treatment area 160 while being moved by the provider over the treatment area, and is further adapted to transmit the images to tracking system processor 130 for processing and building of the node map. Optical reader 131 may be adapted to perform three dimensional (3D) imaging of treatment area 160, or, optionally, two dimensional imaging (2D), using techniques known in the art for laser imaging, ultrasound imaging, video imaging, and/or other forms of optical imaging.

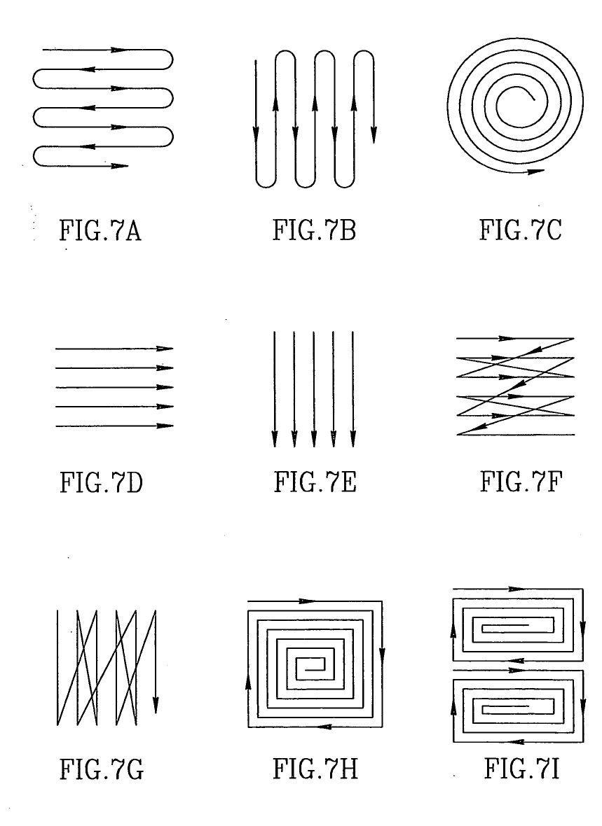

Reference is also made to FIG. 1B which schematically shows the robot arm of FIG. 1A, in greater detail. Robot arm 150 is connected at one end to servomechanism 155 and adapted to move transducer 142 from one node to another node in the node map, following a predetermined raster pattern across treatment area 160. Optionally, movement of transducer 142 follows a predetermined non-raster pattern. Exemplary raster and non-raster patterns are shown in FIG. 7. Robot arm 150 includes two arms 151 and three joints 152, although in some embodiments of the invention, the robot arm may comprise a greater or lesser number of arms and/or joints. Joints 152 are adapted to provide robot arm 150 with capability to translate with up to three degrees of freedom along an x-axis, a y-axis, and/or a z-axis, or any combination thereof. Joints 152 may be further adapted to provide robot arm 150 with capability to translate with other degrees of freedom such as, for example, yaw, roll and/or pitch, or any combination thereof. In some embodiments of the invention, robot arm 150 may be adapted to allow transducer 142 to translate with 1, 2, 3, 4, 5, or 6 degrees of freedom.

Servomechanism 155, responsive to tracking signals received from controller 120, and/or optionally from tracking system processor 130, is adapted to move robot arm 150 finite distances along the x, y, and/or z axes, and optionally roll, yaw and/or pitch, or any combination thereof. Optionally, servomechanism 155 may be further adapted to allow transducer 142 to translate on robot arm 150 with 1, 2, 3, 4, 5, or 6 degrees of freedom. Servomechanism 155 may comprise electric motors, such as, for example, stepper motors, to effect the movement in robot arm 150, and optionally transducer 142. Optionally, in some embodiments of the invention, hydraulic, pneumatic, and/or magnetic means, or any combination thereof, may be used to effect movement in robot arm 150, and optionally transducer 142. Optionally, electric motors may be used in combination with the hydraulic, pneumatic, and/or magnetic means, or any combination thereof.

In accordance with an embodiment of the invention, apparatus 100 is adapted to automatically move transducer 142 from node to node in treatment area 160 and to correctly position the transducer over each node 165. Controller 120, and/or optionally tracking system processor 130, is adapted to control movement of transducer 142 throughout treatment area 160 by controlling, through servomechanism 155, movement of robot arm 150. Controller 120, and/or optionally tracking system processor 130, through servomechanism 155, is further adapted to correctly position transducer 142 over a node 165, optionally by controlling degrees of freedom of translation in transducer 142. Controller 120 routing of transducer 142 throughout treatment area 160 may generally be based on a predetermined raster pattern, although in some embodiments of the invention, routing may be based on a predetermined non-raster pattern. A predetermined routing pattern may be a pattern preprogrammed into controller 120, and/or optionally tracking system processor 130, prior to building of the node map, or may be programmed into the controller during or following building of the node map. Optionally, controller routing of the transducer through the treatment area may be controlled real-time by the treatment provider.

In accordance with an embodiment of the invention, apparatus 100 is adapted to automatically trigger transmissions of FU into each node 165 when transducer 142 is correctly positioned over the node. Controller 120 may compare the real-time position of transducer 142 with respect to node 165, with predetermined criteria for correct positioning over the node. Responsive to equivalence between the actual real-time position and the predetermined criteria, controller 120 sends a trigger signal to FU processing module 140. FU processing module 140, responsive to the trigger signal, powers transducer 142 and triggers transducer transmission of FU.

In accordance with an embodiment of the invention, apparatus 100 is adapted to move transducer 142 iteratively from node to node, and is further adapted to bypass nodes which have received a predetermined amount of transmitted FU. Controller 120 comprises a memory block wherein may be stored a list of nodes which have received the predetermined amount of transmitted FU. Optionally, the memory block may be located externally to the controller. The list may be generally updated every time a node receives the predetermined amount of transmitted FU. As transducer 142 moves across treatment area 160 and stops at every node 165, controller 120 checks if the node does not appear in the memory list. If the node appears, controller 120 guides transducer 142 to the next node. If the node does not appear in the memory list, controller 140 correctly positions transducer 142 over the node and sends a trigger signal to FU processing module 140. In some embodiments of invention, every time controller 120 sends a trigger signal to FU processing module 140, the respective node receiving the acoustic energy is removed from the node map. Consequently, the removed node in the node map becomes essentially non-existent in treatment area 160, and controller 120 guides transducer 142 past the node. Optionally, controller 120 may manage a memory list in the memory block and/or may remove the node from the node map.

In accordance with some embodiments of the invention, apparatus 100 is adapted to continuously move transducer 142 throughout treatment area 160, while the transducer is continuously transmitting FU. Controller 120 is adapted to register the amount of FU transmitted to each node 165, and controls servomechanism 155 so that transducer 142 bypasses nodes which have received the predetermined amount of FU. Optionally, the nodes which have received the predetermined amount of FU are not bypassed, and transducer 142 stops transmitting FU when passing over those nodes.

Optional display 180, keyboard 185 and mouse 182 are adapted to allow treatment provider input/output data interface with apparatus 100. Information may be displayed in display 180 such as, for example, the node map including position of nodes 165, position of transducer 142 within the node map, close up images of nodes in treatment area 160, distant images of treatment area 160, nodes treated in the node map, nodes untreated in the node map, predetermined routing of transducer 142 through treatment area 160, among others. Input data through keyboard 185 and/or mouse 182 may also be displayed in display 180.

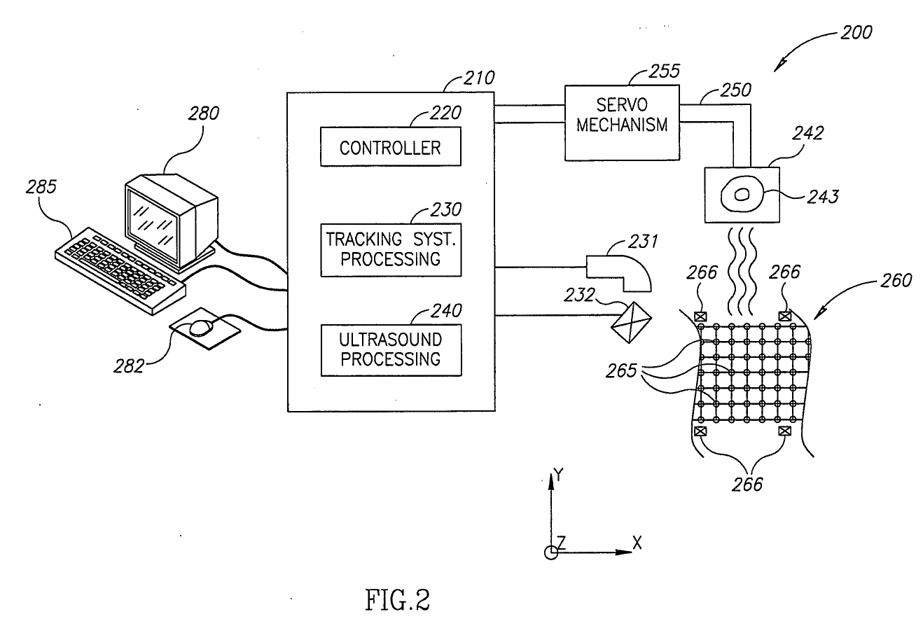

Reference is made to FIG. 2, which schematically shows an exemplary automatic apparatus 200, in accordance with another embodiment of the invention. Apparatus 200 comprises a base unit 210, which includes a controller 220, a tracking system processor 230 and an FU processing module 240; a transducer 242, which connects to base unit 210 through a robot arm 250; a servomechanism 255; an optional display 280; optional data input means such as a keyboard 285 and a mouse 282; and an optional manual optical reader 231. Additionally comprised in apparatus 200 is a tracking system 232. In some embodiments of the invention, tracking system processor 230 may be comprised in controller 220.

Base unit 210, controller 220, tracking system processor 230, FU processing module 240, robot arm 250, servomechanism 255, display 280, keyboard 285, mouse 282, and manual optical imager 231 are the same or substantially similar to that shown in FIG. 1 at 110, 120, 130, 140, 150, 155, 180, 185, 182, and 131. Transducer 242 connects to base unit 210 through robot arm 250, and is substantially similar to transducer 142 shown in FIG. 1, with the exception that transducer 242 does not comprise a tracking system 232. Transducer 242 may include a transducer reference marker 243 adapted to provide a reference to tracking system 232 regarding the position of transducer 242 relative to the treatment area 260. Transducer reference marker 243 may be passive (for example a target image on transducer 242) or active (for example, emitting or irradiating reference signals).

Tracking system 232, which may be functionally similar to that shown in FIG. 1 at 132, is located externally to transducer 242, and positioned in apparatus 200 such that the transducer and treatment area 260, including nodes 265 and reference markers 266, are within tracking and guidance range of the tracking system. Treatment area 260 including nodes 265, and reference markers 266, may be the same or substantially similar to that shown in FIG. 1 at 160, 165 and 166, respectively.

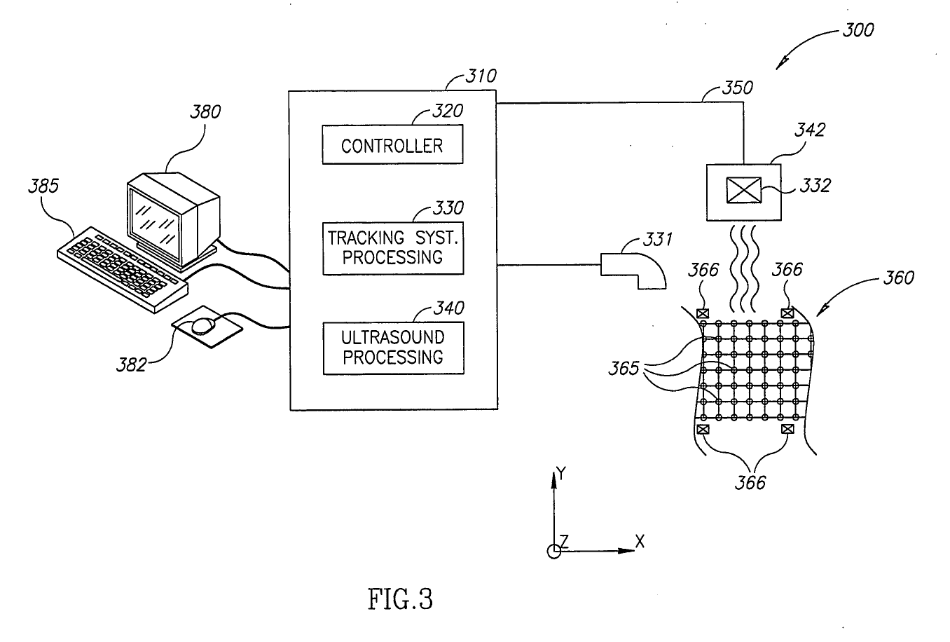

Reference is made to FIG. 3, which schematically shows an exemplary manual apparatus 300, in accordance with another embodiment of the invention. Apparatus 300 comprises a base unit 310, which includes a controller 320, a tracking system processor 330 and an FU processing module 340; a transducer 342, which connects to base unit 310 through a cable, optionally comprised in a mechanical arm 350; an optional display 380; optional data input means such as a keyboard 385 and a mouse 382; and an optional manual optical reader 331. Additionally comprised in apparatus 300 is a tracking system 332. In some embodiments of the invention, tracking system processor 330 may be comprised in controller 320. Base unit 310, tracking system processor 330, FU processing module 340, transducer 332, display 380, keyboard 385, mouse 382, and manual optical reader 331 are the same or substantially similar to that shown in FIG. 1 at 110, 130, 140, 132, 180, 185, 182, and 131.

In accordance with an embodiment of the invention, apparatus 300 is adapted to automatically trigger transmissions of FU into each node 365 in treatment area 360 when transducer 342 is correctly positioned, by the treatment provider, over the node. Treatment area 360 including nodes 365, and reference markers 366, may be the same or substantially similar to that shown in FIG. 1 at 160, 165 and 166, respectively. Controller 320 and/or optionally tracking system processor 330 may compare the real-time position of transducer 342 with respect to node 365, with predetermined criteria for correct positioning over the node. Responsive to equivalence between the actual real-time position and the predetermined criteria, controller 320 sends a trigger signal to FU processing module 340. FU processing module 340, responsive to the trigger signal, powers transducer 342 and triggers transducer transmission of FU. In some embodiments of the invention, apparatus 300 will issue a warning signal prior to triggering of transducer 342. The warning signal may be visually displayed in display 380. Optionally, the warning signal may be an aural signal.

In some embodiments of the invention, apparatus 300 is adapted to track the movement of transducer 342 from node to node, and is further adapted to prevent automatic triggering of the transducer in nodes which have received a predetermined amount of transmitted FU. In some embodiments of the invention, tracking of the movement of transducer 342 may be displayed on display 380, which may display the node map and the position of the transducer in the node map. Controller 320 comprises a memory block wherein may be stored a list of nodes which have received the predetermined amount of transmitted FU. Optionally, the memory block may be located externally to the controller. The list may be generally updated every time a node receives the predetermined amount of transmitted FU. As the provider moves, transducer 342 moves across treatment area 360 and stops at every node; controller 320 checks if the imaged node does not appear in the memory list. If the node appears, controller 320 does not send a trigger signal to FU processing module 340. In some embodiments of the invention, controller 320 may send a warning signal which may be displayed in display 380, and/or optionally activate an aural warning device. If the node does not appear in the memory list, controller 320 sends a trigger signal to FU processing module 340 when the provider correctly positions transducer 342 over the node. In some embodiments of invention, every time controller 320 sends a trigger signal to FU processing module 340, the respective node receiving the acoustic energy is removed from the node map. Consequently, the removed node in the node map becomes essentially non-existent in treatment area 360, and the provider guides transducer 342 past the node. Optionally, controller 320 may manage a memory list in the memory block and/or may remove the node from the node map.

According to some embodiments, treatment area 360 may include a physical node map (not shown) drawn or attached to a treated subject's body. Such physical node map may be used in combination with a tracking system 332 (which may be, for example, a camera) to provide reference of the position of transducer 342 relative to treatment area 360.

In accordance with some embodiments of the invention, apparatus 300 is adapted to continuously transmit FU while transducer 342 is continuously moved by the treatment provider over treatment area 360. Controller 320 is adapted to register the amount of FU transmitted to each node 365, and controls transducer 342 so that it stops transmitting FU when passing over nodes which have received the predetermined amount of FU. Optionally, controller 320 may send a warning signal which may be displayed in display 380, and/or optionally activate an aural warning device.

Mechanical arm 350 may be substantially similar to robot arm 150 shown in FIG. 1B with the exception that the mechanical arm is adapted to support transducer 342 such that transducer 342 may be moved by the treatment provider from one node 365 to another node in the node map. Mechanical arm 350 may comprise translation with up to three degrees of freedom along an x-axis, a y-axis, and/or a z-axis, or any combination thereof. Optionally, mechanical arm 350 may comprise translation with other degrees of freedom such as, for example, yaw, roll and/or pitch, or any combination thereof. In some embodiments of the invention, mechanical arm 350 may be adapted to allow transducer 342 to translate with 1, 2, 3, 4, 5, or 6 degrees of freedom.

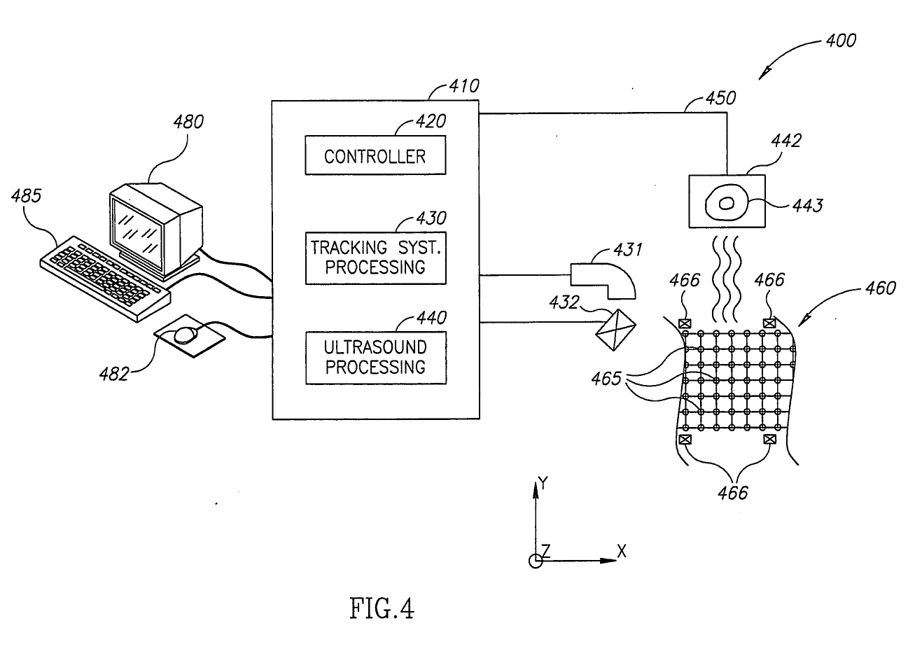

Reference is made to FIG. 4, which schematically shows an exemplary manual apparatus 400, in accordance with another embodiment of the invention. Apparatus 400 comprises a base unit 410, which includes a controller 420, a tracking system processor 430 and an FU processing module 440; a transducer 442 which connects to base unit 410 through a cable, optionally comprised in a mechanical arm 450; an optional display 480; optional data input means such as a keyboard 485 and a mouse 482; and an optional manual optical reader 431. Additionally comprised in apparatus 400 is a tracking system 432. In some embodiments of the invention, tracking system processor 430 may be comprised in controller 420.

Base unit 410, controller 420, tracking system processor 430, FU processing module 440, mechanical arm 450, display 480, keyboard 485, mouse 482, and manual optical reader 431 may be the same or substantially similar to that shown in FIG. 3 at 310, 320, 330, 340, 350, 380, 385, 382, and 331. Transducer 442 connects to base unit 410 through mechanical arm 450, and is substantially similar to transducer 342 shown in FIG. 3 with the exception that transducer 442 does not comprise a tracking system 432. Optionally, mechanical arm 450 may be substantially similar to that optionally shown in FIG. 3 at 350.

Tracking system 432, which may be functionally similar to that shown in FIG. 3 at 332, is located externally to transducer 442, and positioned in apparatus 400, such that the transducer and treatment area 460, including nodes 465 and reference markers 466, are within tracking range of the tracking system. Treatment area 460 including nodes 465, and reference markers 466, may be the same or substantially similar to that shown in FIG. 3 at 360, 365 and 366, respectively. Transducer 442 further includes a transducer reference marker 443 adapted to provide a reference to tracking system 432 regarding the position of transducer 242 relative to treatment area 460. Transducer reference marker 443 may be passive (for example a target image on transducer 442) or active (for example, emitting or irradiating reference signals).

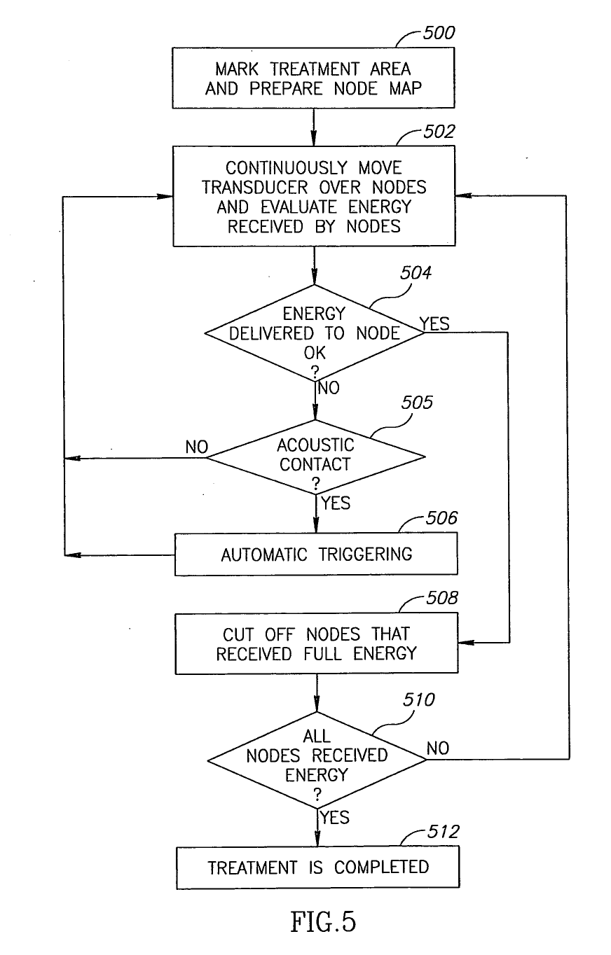

Reference is made to FIG. 5, which shows a flowchart of an exemplary “continuous” mode of operation of apparatus 100 shown in FIG. 1, or optionally, apparatus 200 shown in FIG. 2, or optionally apparatus 300 shown in FIG. 3 or apparatus 400 shown in FIG. 4, in accordance with an embodiment of the invention. The mode of operation described is not intended to be limiting, and it may be clear to a person skilled in the art that other combinations and/or sequences of steps may be used when operating the apparatus.

[STEP 500] The treatment area is prepared and reference markers are positioned. Marker may be of the passive type or active type, depending on the type of tracking system comprised in the apparatus. The tracking system is calibrated relative to the position of the markers. Registration (synchronization) of the tracking system is performed. Optionally, the treatment area may be manually scanned by the provider, using the manual optical reader. The node map is then built by the controller, and/or optionally the tracking system processor, in the apparatus. Optionally, the node map may be manually built by the treatment provider.

In an embodiment of the invention, the apparatus is adapted to route the movement of the transducer through the treatment area, based on a preprogrammed algorithm, for example, an algorithm comprising a raster pattern, or optionally, a non-raster pattern. In some embodiments of the invention, the treatment provider may determine the routing to be followed by the transducer. For example, the node map may be displayed on the display and the user using the mouse may define the route by tracing the path of the transducer from one node to another, until all nodes in the node map are covered.

[STEP 502] The transducer, responsive to control signals sent from the controller to the servomechanism module, is moved by the robot arm to a node, according to the predetermined route. The controller checks if the node appears in the memory list. Optionally, the transducer is moved by a manual movement of the mechanical arm to the node (technician moves the mechanical arm), according to the predetermined route.

[STEP 504] If the node appears in the memory list, go to STEP 508. If the node does not appear in the memory list, go to STEP 505.

[STEP 505] Verify acoustic contact. If there is acoustic contact, go to STEP 506. If there is no acoustic contact, go to STEP 502.

[STEP 506] The controller, through the servomechanism module, moves the robot arm, and optionally the transducer, so as to correctly position the transducer over the node. Optionally, the transducer is positioned over the node by manual movement of the mechanical arm. Upon comparing received real-time transducer position relative to the node, with the predetermined criteria for correct positioning, the controller sends a trigger signal to the FU processing module. The FU processing module, in response, sends a power signal, and a trigger signal, to the transducer, triggering the transducer to transmit FU to the node. The node is registered in the memory list. Following transmission of FU, return to STEP 502. [STEP 508]

The controller sends a warning signal, which may be displayed or sounded in the apparatus advising of the node having received a predetermined amount of FU. Automatic triggering of the transducer is prevented. In some embodiments of the invention, the node may be removed from the node map to prevent the transducer from stopping at the node again.

[STEP 510] The controller checks if all nodes appear in the memory list. If no, go to STEP 502. If yes, go to STEP 512.

[STEP 512] Treatment is completed.

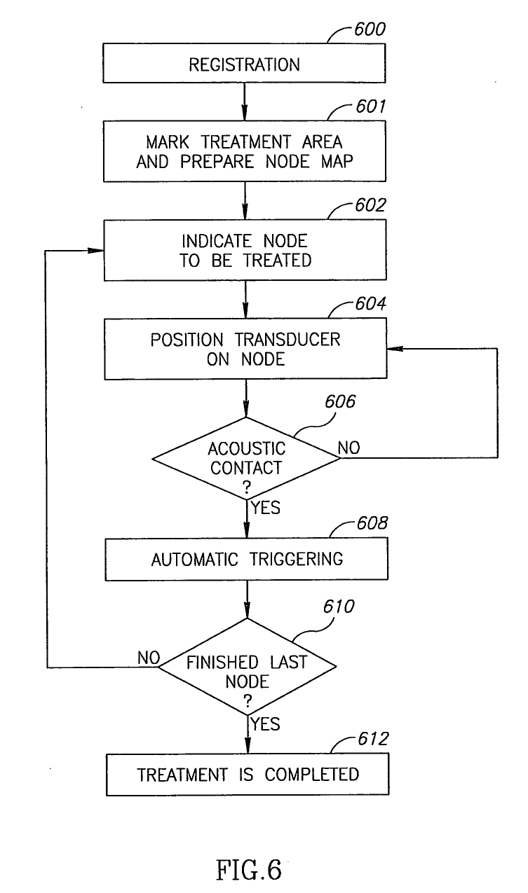

Reference is made to FIG. 6, which shows a flowchart of an exemplary mode of operation of apparatus 100 shown in FIG. 1 or apparatus 200 shown in FIG. 2, or optionally apparatus 300 shown in FIG. 3 or apparatus 400 shown in FIG. 4, in accordance with an embodiment of the invention. The mode of operation described is not intended to be limiting, and it may be clear to a person skilled in the art that other combinations and/or sequences of steps may be used when operating the apparatus.

[STEP 600] The treatment area is prepared and reference markers are positioned. Markers may be of the passive type or active type depending on the type of tracking system comprised in the apparatus. The tracking system is calibrated relative to the position of the markers, and registration (synchronization) of the tracking system is performed. Optionally, the treatment area is manually scanned by the treatment provider using the manual optical reader.

[STEP 601] The node map is built by the controller, and/or optionally the tracking system processor, in the apparatus. Optionally, the node map may be manually built by the treatment provider.

In an embodiment of the invention, the apparatus is adapted to route the path to be followed by the treatment provider moving the transducer through the treatment area based on a preprogrammed algorithm, for example, an algorithm comprising a raster pattern, or optionally, a non-raster pattern. In some embodiments of the invention, the provider may determine the routing to be followed by the transducer. For example, the node map may be displayed on the display and the provider, using the mouse, may define the route by tracing the path of the transducer from one node to another, until all nodes in the node map are covered. Optionally, the provider may randomly select the nodes. For example, after a node is treated, the provider selects the next node to be treated from the node map.

[STEP 602] The node to be treated may be displayed to the provider on the display. Optionally, the provider may select the node to be treated.

[STEP 604] The provider moves the transducer to the selected node.

[STEP 606] The controller compares the received real-time transducer position relative to the node, with the predetermined criteria for correct positioning, and checks for acceptable acoustic contact. If an acceptable acoustic contact is not formed go to STEP 604.

[STEP 608] If positioning is correct, the controller sends a trigger signal to the FU processing module. The FU processing module, in response, sends a power signal and a trigger signal to the transducer, triggering the transducer to transmit FU to the node. The node is registered in the memory list of the controller.

[STEP 610] The controller checks if all nodes appear in the memory list. If no, go to STEP 602. Optionally, the treatment provider may check the memory list of the controller. Additionally or alternatively, the node may be removed from the node map. The provider may then check the node map to see if there are any nodes left.

[STEP 612] Treatment is completed.

Reference is made to FIGS. 7A-71, which schematically show exemplary raster and non-raster patterns which may be followed by the transducer shown in FIG. 1, and optionally the transducers of FIGS. 2, 3 and/or 4, when moving, automatically or manually, throughout the treatment area, in accordance with some embodiments of the invention. The patterns shown are not intended to be limiting, and it may be clear to a person skilled in the art that other patterns may be followed when operating the apparatus.

FIG. 7A shows a horizontal, top-bottom, left-right raster pattern, although optionally, the pattern may be right-left, and/or bottom-top, in accordance with some embodiments of the invention.

FIG. 7B shows a vertical top-bottom, left-right raster pattern, although optionally, the pattern may be right-left, and/or bottom-up, in accordance with some embodiments of the invention.

FIG. 7C shows a spiral pattern starting from a center of the spiral and leading away from the center in a counterclockwise direction, in accordance with some embodiments of the invention. Optionally, the spiral may be in a clockwise direction. Optionally, the spiral may start from outside and lead to towards the center.

FIG. 7D shows a pattern comprising parallel, straight lines in a horizontal left-right direction, in accordance with some embodiments of the invention. Optionally, the lines may be in a right-left direction.

FIG. 7E shows a pattern comprising parallel, straight lines in a vertical top-bottom direction, in accordance with some embodiments of the invention. Optionally, the lines may be in a bottom-top direction.

FIG. 7F shows a pattern comprising parallel lines in a horizontal left-right direction intersected by diagonal lines, in accordance with some embodiments of the invention. Optionally, the horizontal lines may be in a right-left direction.

FIG. 7G shows a pattern comprising parallel lines in a vertical top-bottom direction intersected by diagonal lines, in accordance with some embodiments of the invention. Optionally, the vertical lines may be in a bottom-top direction.

FIG. 7H shows a rectangular spiral pattern, which leads from an outside towards a center of a rectangle, starting from a left-to-right direction, in accordance with some embodiments of the invention. Optionally, the rectangular spiral pattern may start from the center and lead outwards. Optionally, the rectangular spiral may start from a right-to-left direction.

FIG. 7I shows two rectangular spiral patterns, each leading from an outside towards a center of each rectangle, starting from a left-to-right direction, in accordance with some embodiments of the invention. Optionally, the rectangular spiral patterns may start from the center and lead outwards. Optionally, the rectangular spirals may start from a right-to-left direction. Optionally, each rectangular spiral may start from a different direction and/or may lead in a different direction.

Reference is made to FIG. 8, which schematically shows an exemplary ultrasonic acoustic energy signal transmitted by the transducer shown in FIG. 1, and optionally the transducers of FIGS. 2, 3 and/or 4, in accordance with some embodiments of the invention. The signal may include an essentially sinusoidal waveform comprising the following characteristics;

f=frequency of the signal;

T=period of the signal, 1/f;

Ton=duration of a burst (burst length);

Tbrp=burst repetition period (time interval between two successive bursts);

DC=duty cycle, Ton/Tbrp;

BRF=burst repetition frequency, 1/Tbrp; and

Tn=treatment time per node (transmission time or exposure time, per node).

FIG. 8 is shown herein for purposes of elucidation and simplification. It is noted that FIG. 8 shows only an example and the number of periods in a burst, and is not limited to three in each pulse of duration Tn (as it is shown in FIG. 8) but can be any number, such as 2-10, or any other appropriate number.

In the description and claims of embodiments of the present invention, each of the words, “comprise” “include” and “have”, and forms thereof, are not necessarily limited to members in a list with which the words may be associated.

The invention has been described using various detailed descriptions of embodiments thereof that are provided by way of example and are not intended to limit the scope of the invention. The described embodiments may comprise different features, not all of which are required in all embodiments of the invention. Some embodiments of the invention utilize only some of the features or possible combinations of the features. Variations of embodiments of the invention that are described and embodiments of the invention comprising different combinations of features noted in the described embodiments will occur to persons with skill in the art.

What is claimed is:

1. An apparatus for lysing of adipose tissue comprising:
a transducer adapted to transmit ultrasound acoustic waves to a target area tissue of a subject body; and
a controller adapted to automatically trigger the transducer to transmit ultrasound acoustic waves upon receiving indication of said transducer being positioned at a predetermined position (node).

2. The apparatus according to claim 1, wherein indication of said transducer being positioned at a predetermined position is provided by a tracking system.

3. The apparatus according to claim 2, wherein the tracking system comprises a node map.

4. The apparatus according to claim 1, further comprising a positioning element adapted to automatically position the transducer at the predetermined position.

5. The apparatus according to claim 4, wherein the positioning element comprises a robot arm.

6. The apparatus according to claim 4, wherein the positioning element provides at least two degrees of freedom of movement.

7. The apparatus according to claim 1, wherein said transducer is adapted to transmit ultrasound acoustic waves with a duty cycle in the range of 1:3-1:400.

8. The apparatus according to claim 1, wherein said transducer is adapted to transmit ultrasound acoustic waves at a pulse repetition frequency (PRF) range of 20-1100 Hz.

9. An apparatus for lysing of adipose tissue comprising:
a transducer adapted to transmit ultrasound acoustic waves to a target area tissue of a subject body; and
a positioning element adapted to automatically position the transducer at a predetermined position (node).

10. The apparatus according to claim 9, further comprising a controller adapted to notify a user upon receiving indication of said transducer being positioned at a predetermined position (node).

11. The apparatus according to claim 9, further comprising a controller adapted to automatically trigger the transducer to transmit ultrasound acoustic waves upon receiving indication of said transducer being positioned at a predetermined position.

12. The apparatus according to claim 9, wherein the positioning element comprises a robot arm.

13. The apparatus according to claim 9, wherein the positioning element provides at least two degrees of freedom of movement.

14. The apparatus according to claim 9, wherein the positioning element is adapted to continuously move the transducer at a predefined speed.

15. The apparatus according to claim 14, wherein the rate of movement is adapted to vary during a treatment.

16. The apparatus according to claim 14, wherein the positioning element is adapted to continuously move the transducer in accordance with a predetermined path.

17. The apparatus according to claim 9, further comprising a processor adapted to compute the amount of ultrasonic energy transmitted to a node of interest.

18. The apparatus according to claim 9, further comprising a controller adapted to initiate said positioning element to return to a specific node upon receiving an indication that the amount of ultrasonic energy transmitted to said specific node is below a predetermined threshold.

19. The apparatus according to claim 9, wherein the positioning element is adapted to automatically position the transducer at a predetermined position based on data obtained by a tracking system.

20. The apparatus according to claim 9, wherein the tracking system comprises a node map.

21. A system for lysing of adipose tissue comprising:
a transducer adapted to transmit ultrasound acoustic waves to a target area tissue of a subject body;
a positioning element adapted to automatically position said transducer at a predetermined position; and
a controller adapted to automatically trigger said transducer to transmit ultrasound acoustic waves upon receiving indication of said transducer being positioned at the predetermined position.

22. A method for lysing of adipose tissue comprising:
positioning an ultrasonic transducer at a predetermined position on or in proximity to a treatment area of a subject body; and
automatically triggering the transducer to transmit ultrasound acoustic waves upon receiving indication of the transducer being at the predetermined position (node).

23. The method according to claim 22, wherein positioning the ultrasonic transducer comprises manually positioning.

24. The method according to claim 22, wherein positioning the ultrasonic transducer comprises automatically positioning.

25. The method according to claim 22, further comprising continuously moving the transducer at a predefined rate.

26. The method according to claim 25, wherein the rate of movement is adapted to vary during a treatment.

27. The method according to claim 25, wherein the transducer is continuously moving in accordance with a predetermined path.

28. The method according to claim 22, further comprising computing the amount of ultrasonic energy transmitted to a node of interest.

29. The method according to claim 28, further comprising returning to a specific node upon receiving indication that the amount of ultrasonic energy transmitted to the specific node is at or below a predetermined threshold and automatically triggering the transducer to transmit ultrasound acoustic waves.

30. A method for lysing of adipose tissue comprising:
positioning an ultrasonic transducer at a predetermined position (node) on or in proximity to a treatment area of a subject body;
triggering the transducer to transmit ultrasound acoustic waves upon receiving indication of the transducer being at the predetermined position (node);
computing the amount of ultrasonic energy transmitted to a node of interest; and
returning to a specific node upon receiving indication that the amount of ultrasonic energy transmitted to the specific node is at or below a predetermined threshold and triggering the transducer to transmit ultrasound acoustic waves.

31. The method according to claim 30, wherein positioning the ultrasonic transducer comprises manually positioning.

32. The method according to claim 30, wherein positioning the ultrasonic transducer comprises automatically positioning.

33. The method according to claim 30, wherein triggering comprises automatic triggering.
